(19) United States
(12) Patent Application Publication
(10) Pub. No.: US 2004/0266864 A1
(43) Pub. Date: Dec. 30, 2004

(54) Methods for treating depression and other CNS disorders using enantiomerically enriched desmethyl-and didesmethyl-metabolites of citalopram

(75) Inventors: Larry R. Bush, Worcester, MA (US); Mark G. Currie, Sterling, MA (US); Q. Kevin Fang, Wellesley, MA (US); Chris H. Senanayake, Brookfield, CT (US)

Correspondence Address: HESLIN ROTHENBERG FARLEY & MESITI PC, 5 COLUMBIA CIRCLE, ALBANY, NY, 12203, US

(73) Assignee: Sepracor Inc., Marlborough, MA

(21) Appl. No.: 10/842,055

(22) Filed: May 7, 2004

Related U.S. Application Data
(63) Continuation of application No. PCT/US02/35408, filed on Nov. 5, 2002.
(60) Provisional application No. 60/337,608, filed on Nov. 8, 2001.

(51) Int. Cl. C07D 307/78; A61K 31/343

(52) U.S. Cl. 514/469; 549/467

(57) ABSTRACT

This invention relates to novel compositions of matter containing enantiomerically enriched (−)-desmethylcitalopram, (+)-didesmethylcitalopram, or (−)-didesmethylcitalopram or mixtures thereof in optimal ratios. Contrary to prior teachings, the enantiomerically enriched citalopram metabolites disclosed herein possess potent serotonin reuptake inhibitory activity, with minimal inhibitory effects on the reuptake of other known monoamines, e.g., norepinephrine (NE) or dopamine (DA). The present invention also discloses methods for treating disorders, dysfunctions and diseases for which inhibition of serotonin reuptake is therapeutically beneficial. In particular, the present invention discloses a method for treating various forms of depression with pharmaceutical compositions described herein.

### CROSS REFERENCE TO RELATED APPLICATIONS

This application is a continuation of copending International Application No. PCT/US02/35408, filed on Nov. 5, 2002, published under International Publication Number WO 01/040121 on 15 May 2003, and claims priority from U.S. provisional application Ser. No. 60/337,608, filed on Nov. 8, 2001. The entire disclosures of both are incorporated herein by reference.

### BACKGROUND OF THE INVENTION

Citalopram 1 (CIT), an antidepressant of the selective serotonin reuptake inhibitor (SSRI) type, is one of the most selective of the SSRIs. As an SSRI, citalopram has been used to treat CNS affective disorders such as depression, as well as indications wherein inhibition of serotonin reuptake is desired. These indications include anxiety, obsessive-compulsive disorders, various phobias, borderline personality disorders, and bipolar disorders. Citalopram has also been used for treatment of alcohol and tobacco substance abuse or addictions. Citalopram possesses one stereocenter, and therefore exists in (+) and (−) forms. Racemic citalopram is commercially available in the US under the trade name CELEXA.

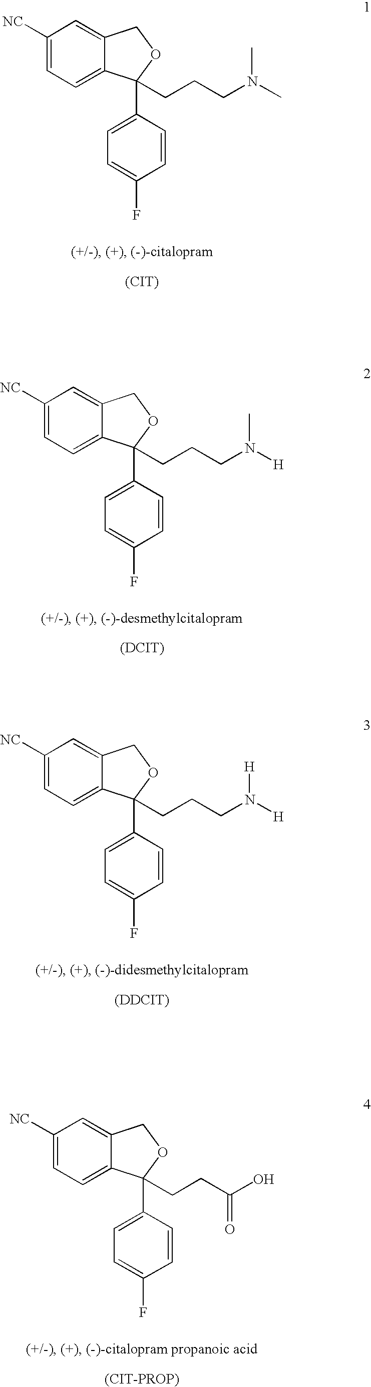

In humans, biotransformation of citalopram has been attributed (in vitro) to the specific human hepatic cytochrome enzymes P450 4A, P450 2C19 and, to a minimal extent, P450 2D6 (Willets, 1999). Citalopram is stereoselectively metabolized in the liver to the polar metabolites, partially by N-demethylation to desmethylcitalopram 2 (DICT) and didesmethylcitalopram 3 (DDCIT), as well as by deamination to a propanoic acid metabolite 4 (CIT-PROP) and by N-oxidation to CIT-N-oxide (Baumann et al, 1995). Importantly, neither citalopram nor its metabolite desmethylcitalopram inhibit the activity of these or other cytochrome P450 enzymes (P450 1A2, P450 2C9, P450 2E1) by more than a mild degree. Citalopram's negligible affinity for receptors for various neurotransmitters (e.g., acetylcholine, histamine, norepinephrine, and dopamine), enzymes (e.g., monoamine oxidase), and other reuptake sites (dopamine and norepinephrine) is thought to account for its relative safety and tolerability, as well as its growing popularity among physicians prescribing antidepressants (Willets, 1999). Furthermore, citalopram's negligible effects on P450 enzymes contribute to the drug's safety in view of drug-drug interactions with other substrates.

### SUMMARY OF THE INVENTION

The present invention relates to novel compositions of matter containing enantiomerically enriched (−)-desmethylcitalopram, (−)- and (+)-didesmethylcitalopram or mixtures thereof or metabolites thereof in optimal ratios. Such compositions possess potent serotonin reuptake inhibitory activity, with minimal inhibitory effects on the reuptake of other known monoamines, e.g., norepinephrine (NE) or dopamine (DA). Although (−)-desmethylcitalopram possesses less serotonin reuptake inhibitory property than its parent citalopram, the data disclosed herein clearly indicate that both it and its immediate downstream metabolite, (−)- and (+)-didesmethylcitalopram, retain considerable serotonin reuptake inhibitory activity. Furthermore, their half-lives are measurably longer compared to citalopram (Sanchez and Hyttel 1999). These pharmacokinetic properties could make (−)-CIT, (−)-DCIT, (+)-DDCIT or (−)-DDCIT more appropriate for long-term therapy than the racemic or single enantiomer forms of citalopram currently under development.

Reactive, endogenous, and manic depressions are CNS affective disorders for which serotonin reuptake inhibitors are particularly effective. Accordingly the present invention discloses a method for treating depression and CNS affective disorders with pharmaceutical compositions described herein. The methods described herein are also useful for treating or preventing other CNS disorders, cerebrovascular dysfunctions, or vascular dysfunctions, sexual dysfunctions, eating disorders, and substance abuse. The invention also provides a method for co-treatment of the aforementioned disorders, dysfunctions, diseases, or syndromes with antipsychotic, anti-anxiety, or mood-stabilizing agents without compromising the pharmacological/therapeutic effects of the individual pharmaceutical agent in the co-treatment regime. Agents amenable to such a regime include, but are not limited to, clozapine, risperidone, benzodiazepine, or gabapentine.

### DETAILED DESCRIPTION OF THE INVENTION

A. Overview

The present invention discloses compositions and methods for treating disease states associated with serotonergic dysfunctions. In particular the present invention relates to novel compositions of matter containing enantiomerically enriched (−)-desmethylcitalopram, (−)- and (+)-didesmethylcitalopram or mixtures thereof or metabolites thereof in optimal ratios. The present invention also discloses methods for treating affective disorders, namely depression. Additionally, the present invention discloses methods for treating disease states where serotonin reuptake inhibition would be beneficial. These disease states include disorders or dysfunctions of the CNS, cerebrovascular, or vascular systems.

B. Definitions

As used herein, the terms “citalopram” or “CIT” mean the racemic compound shown in Formula 1, which is chemically known as (±)-1-(3-dimethylaminopropyl)-1-(4-fluorophenyl)-1,3-dihydroisobenzofuran-5-carbonitrile.

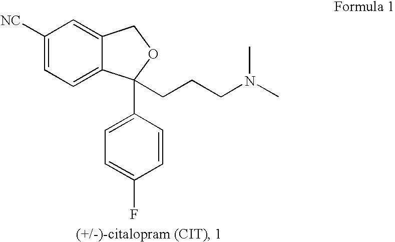

As used herein, the terms “desmethylcitalopram” or “DCIT” mean the racemic or enantiomerically enriched compound shown in Formula 2 which is chemically known as (±)-1-(3-methylaminopropyl)-1-(4-fluorophenyl)-1,3-dihydroisobenzofuran-5-carbonitrile.

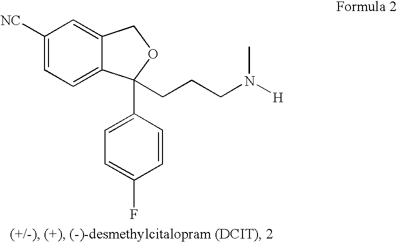

As used herein, the terms “didesmethylcitalopram” or “DDCIT” mean the racemic or enantiomerically enriched compound shown in FIG. 3 which is chemically known as (±)-1-(3-aminopropyl)-1-(4-fluorophenyl)-1,3-dihydroisobenzofuran-5-carbonitrile.

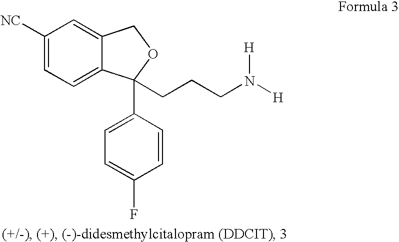

As used herein, the terms “citalopram propanoic acid” or “CIT-PROP” mean the racemic or enantiomerically enriched compound shown in Formula 4 which is chemically known as (±)-1-(3-propanoic)-1-(4-fluorophenyl)-1,3-dihydroisobenzofuran-5-carbonitrile.

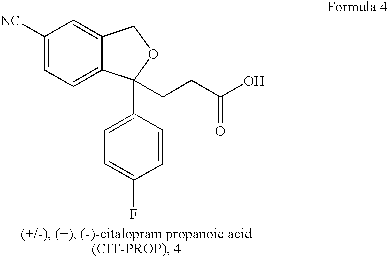

As used herein the term “citalopram metabolites” encompasses, but is not limited to, mammalian metabolites of racemic citalopram. In particular, the term “citalopram metabolites” includes (±) desmethylcitalopram, (±) didesmethylcitalopram, or citalopram propanoic acid.

As used herein, the term “affective disorder” refers to a mental disorder characterized by a disturbance in the regulation of mood, behavior and affect. This disorder includes, but is not limited to, depression, anxiety disorders, attention deficit disorder, attention deficit disorder with hyperactivity, bipolar and manic conditions, bulimia, obesity or weight gain, narcolepsy, chronic fatigue syndrome, seasonal affective disorder, premenstrual syndrome, substance addiction or abuse, and nicotine addiction. A major feature of the clinical picture of affective disorders is dysphoric mood or pervasive loss of interest or pleasure, accompanied by a number of the following symptoms: sleep and appetite disturbances, loss of energy, diminishment of sex drive, onset of body aches or pains, memory loss, inability to make decisions, feelings of self-reproach or excessive or inappropriate guilt, suicidal thoughts, and reduced ability to concentrate.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

As used herein, the term “a method for treating vascular disorders” means relief from the disorders of the vascular system including, but not limited to, myocardial infarction, angina, stroke, pulmonary embolism, transient ischemic attack, deep vein thrombosis, thrombotic re-occulusion subsequent to a coronary intervention procedure, heart surgery or vascular surgery, peripheral vascular thrombosis, Syndrome X, heart failure, or a disorder in which a narrowing of at least one coronary artery occurs.

As used herein, the term “a method of treating serotonergic dysfunctions” means relief from symptoms of a disruption of serotonin neurotransmission manifesting in neurodegenerative diseases as well as other diseases affecting spinal and supraspinal regulation of motor control, the central nervous system including regulation of sensory, autonomic, cognitive, and affective functions, vascular system, cerebrovascular systems or the integrity of the blood brain-barrier.

As used herein, the term “a method of treating depression” means relief from the symptoms of depression which include, but are not limited to, changes in mood, feelings of intense sadness, despair, mental slowing, loss of concentration, pessimistic worry, agitation, and self-deprecation. Physical changes may also be relieved, including insomnia, anorexia, weight loss, decreased energy and libido, and abnormal hormonal circadian rhythms.

As used herein, the term “a method for treating sexual dysfunction” relief from symptoms of including, but not limited to loss of sexual desire, sexual arousal disorder, inability to obtain or maintain an erection, premature ejaculation, absence of emission, or inability to achieve erection correlated with endocrine, drug, local, neurologic, or vascular causes.

As used herein, the term “a method for treating cerebral function disorders” means relief from disease states associated with cerebral function disorders involving intellectual deficits which include, but are not limited to, senile dementia, Alzheimer's type dementia, memory loss, amnesia/amnestic syndrome, disturbances of consciousness, coma, lowering of attention, speech disorders, Parkinson's disease, Lennox syndrome, autism, hyperkinetic syndrome and schizophrenia. Also within the meaning of cerebral function disorders are disorders caused by cerebrovascular diseases including, but not limited to, cerebral infarction, cerebral bleeding, cerebral arteriosclerosis, cerebral venous thrombosis, head injuries, and the like and where symptoms include disturbances of consciousness, senile dementia, coma, diminished of attention, speech disorders, and the like.

The terms “substance abuse”, “pre-menstrual syndrome”, “anxiety”, “panic disorder endogenous depression”, “sleep disorders”, “borderline personality disorders”, “post-traumatic stress disorders”, and “eating disorders” are used herein in a manner consistent with their accepted meanings in the art. See, e.g., Diagnostic and Statistical Manual of Mental Disorders, 4^{th} Ed., American Psychiatric Association, (1997) (DSM-IV™). The terms “method of treating or preventing”, “method of treating”, and “method of preventing” when used in connection with these disorders mean the amelioration, prevention, or relief from the symptoms and/or effects associated with these disorders. Without being limited by any theory, the treatment or prevention of certain of these disorders may be related to the activity of the active ingredient(s) as inhibitors of serotonin reuptake.

The term “healthcare providers” refers to individuals or organizations that provide healthcare services to a person, community, etc. Examples of “healthcare providers” include doctors, hospitals, continuing care retirement communities, skilled nursing facilities, subacute care facilities, clinics, multispecialty clinics, freestanding ambulatory centers, home health agencies, and HMO's.

The term “as valence and stability permits” in reference to compounds disclosed herein refers to compounds that have in vitro or in vivo half-lives at room temperature of at least 12 hours, or at least 24 hours, and are preferably capable of being stored at 0° C. for a week without decomposing by more than about 10%.

The term “clathrate” refers to inclusion compounds in which the guest molecule is in a cage formed by the host molecule or by a lattice of host molecules.

As used in this disclosure, “enantiomerically enriched” refers to products whose enantiomeric excess is greater than zero. Enantiomeric excess (ee) is the “excess” of one enantiomer over the other. As a result, since both enantiomers are present in equal amounts in a racemic mixture, the enantiomeric excess would be zero (0% ee). However, if one enantiomer were enriched such that it constitutes 95% of the product, then the enantiomeric excess would be 90% ee (95%-5% (the amount of the enriched enantiomer minus the amount of the other enantiomer)). In general, higher enantiomeric purity (>about 50% ee) is preferred, with enantiomeric excess of between about 75% ee and about 90% ee being more preferred, and enantiomeric excess of greater than about 90% ee being particularly preferred.

The term “enantiomerically pure” refers to a compound wherein the enantiomeric excess is about 100%.

The terms “half-life” or “half-lives” refer to the time required for half of a quantity of a substance to be converted to another chemically distinct species in vitro or in vivo.

The term “metabolic derivative” refers to a compound derived by one or more in vitro or in vivo enzymatic transformations on the parent compound, wherein the resulting derivative has an ED₅₀ value as a serotonin reuptake inhibitor that is less than 1000×ED₅₀ value of the parent compound. The term “ED₅₀” means the dose of a drug that produces 50% of its maximum response or effect.

The term “prodrug” refers to any compound that is converted to a more pharmacologically active compound under physiological conditions (i.e., in vivo). A common method for making a prodrug is to select moieties that are hydrolyzed under physiological conditions to provide the desired biologically active drug.

The abbreviations Me, Et, Ph, Tf, Nf, Ts, Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

Certain compounds of the present invention may exist in particular geometric or stereoisomeric forms. The present invention contemplates all such compounds, including cis- and trans-isomers, (−)- and (+)-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts may be formed with an appropriate optically active acid or base, followed by resolution of the diastereomers thus formed by fractional crystallization or chromatographic means well known in the art, and subsequent recovery of the pure enantiomers.

As herein used, the term “chiral ligand” is well known in the art and means one or more chiral chemical compounds of organic, inorganic or organometallic nature that is present in a reaction covalently or ionically bonded to one or more reagents, or is a catalytic or quantitative reagent on its own, designed to facilitate enantiomeric excess induction in a reaction which would otherwise produce racemic products. Known ligands suitable for the present invention include, but are not limited to, substituted or unsubstituted binaphthyls, alkylphosphines, arylphosphines, aziridines, diols, amino alcohols, alkylpyrrolidines, aryl pryrrolidines, diamines, aminoacids, carbohydrates, oxazolines, or phosphinoalkyloxazolines.

Contemplated equivalents of the compounds described above include compounds which otherwise correspond thereto, and which have the same general properties thereof (e.g., the ability to inhibit serotonin reuptake), wherein one or more simple variations of substituents are made which do not adversely affect the efficacy of the compound. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover. Also for purposes of this invention, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds which can be substituted or unsubstituted.

C. Exemplary Embodiments

The invention relates to racemic or enantiomerically enriched pharmaceutical compositions of a citalopram metabolite other than (+)-desmethylcitalopram. In particular the invention relates to racemic or enantiomerically pure pharmaceutical compositions of (−)-desmethylcitalopram, or enantiomerically enriched (+)-didesmethylcitalopram or (−)-didesmethylcitalopram. This invention further relates to the synthesis of racemic or enantiomerically pure or enriched citalopram metabolites and to compositions (e.g. pharmaceutical compositions) comprising them. The invention also relates to novel uses of the compounds disclosed herein, which constitute improvements over the use of the racemic citalopram as well as improvements over the optically pure (+) isomer of citalopram.

One embodiment of the invention relates to compounds having structures depicted in Formula I.

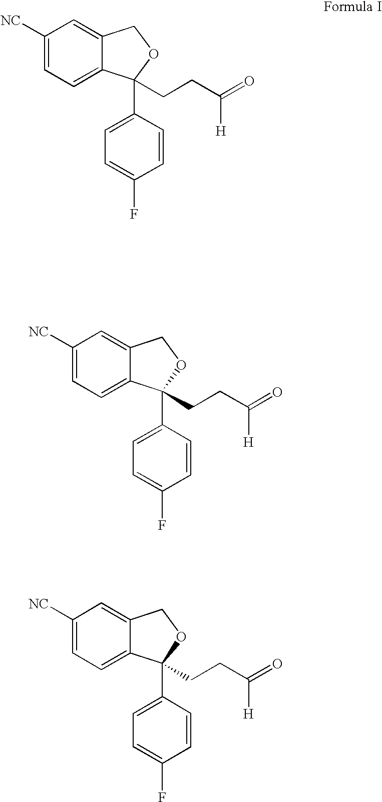

Another embodiment of the invention relates to compounds having structures depicted in Formula II:

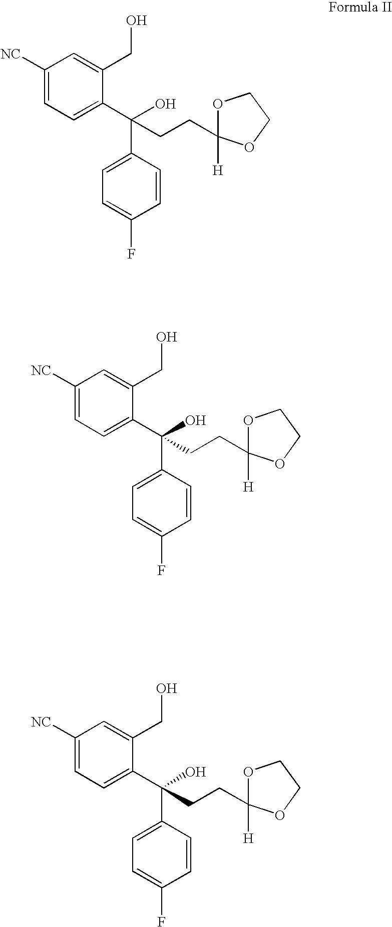

In one embodiment of the invention, the composition comprises, individually or in combination, (−)-desmethylcitalopram, (−)-didesmethylcitalopram, (+)-didesmethylcitalopram. In one embodiment, the amount of the (−)-desmethyl, (−)-didesmethyl, (+)-didesmethyl metabolite of citalopram comprises greater than about 1%, 5%, 10%, 25%, 50%, 75%, or even 90% by weight of the composition.

Another aspect of the invention encompasses a method for preparing racemic desmethylcitalopram which comprises contacting, preferably sequentially, the commercially available phthalide with two Grignard reagents, followed by effecting ring closure, acidic hydrolysis, and a subsequent reductive amination step, as shown in Scheme 1. The preferred Grignard reagents are 4-fluorophenyl magnesium bromide and ethyldioxolane magnesium bromide. In certain embodiments, the reagent used to effect ring closure is mesyl chloride. The preferred reagents for reductive amination are methylamine and sodium borohydride. In one embodiment of the invention the resultant amine is isolated as is or is reacted with an acid to form a salt or with a metal to form a metal complex.

In one embodiment, the acid used is L-tartaric acid.

In another embodiment of the invention any or all steps of the synthesis are carried out on a solid support or in a combinatorial fashion.

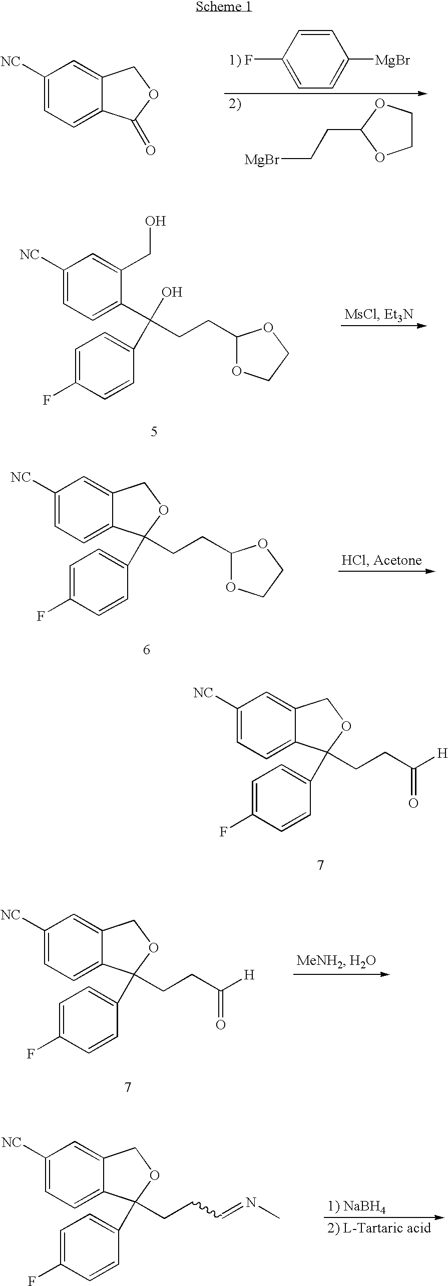

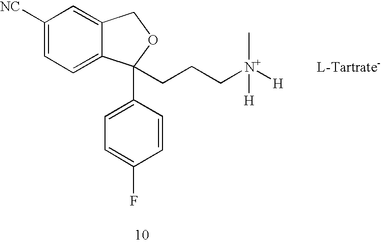

Another embodiment of the invention encompasses a method for preparing enantiomerically enriched (−)-desmethylcitalopram and (+)-desmethylcitalopram which comprises contacting the commercially available phthalide with two Grignard reagents, wherein the second Grignard reagent would use a chiral ligand (by screening many to obtain one with high ee) to give the enantiomerically enriched tertiary alcohol. Ring closure and subsequent reaction are as shown in Scheme 2. In one embodiment of the invention, the resultant amine is isolated as is or is reacted with an acid to form a salt or with a metal to form a metal complex. In one embodiment, the acid used is L-tartaric acid.

In one embodiment of the invention any or all steps of the synthesis are carried out on a solid support or in a combinatorial fashion.

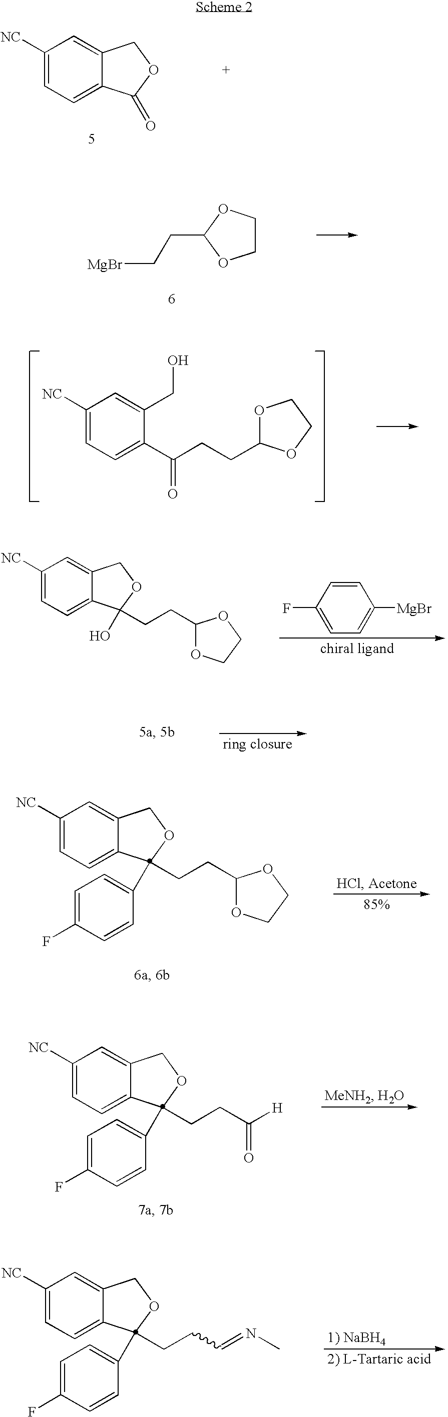

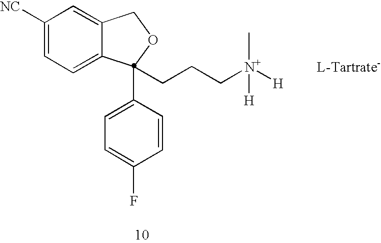

Another embodiment of the invention encompasses a method for preparing enantiomerically enriched (−)-desmethylcitalopram and (+)-desmethylcitalopram including chiral column chromatography resolution of the racemic ketal 6. In one embodiment, a CHIRALCEL OD=column with methanol as eluent is used to resolve the corresponding enantiomers (6a and 6b) as shown in Scheme 3. Subsequent reactions of compound 6a and 6b are the same as in Scheme 2.

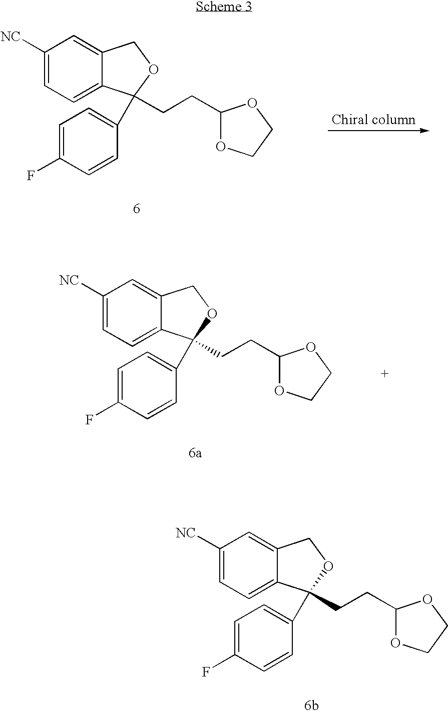

Another embodiment of the invention encompasses a method for preparing racemic didesmethylcitalopram by reductive amination of aldehyde 7 with an ammonia equivalent followed by hydride reduction as shown in Scheme 4. The resultant product can be isolated as a salt. The preferred ammonia equivalent is (−)-tert-butylsulfinamide which is contacted with aldehyde 7 in the presence of an alkoxy titanium reagent. The preferred reductant is sodium borohydride. In one embodiment of the invention, the resultant amine is isolated as is or is reacted with an acid to form a salt or with a metal to form a metal complex. In one embodiment, the acid used to prepare a salt is L-tartaric acid. In one embodiment of the invention, any or all steps of the synthesis are carried out on a solid support or in a combinatorial fashion.

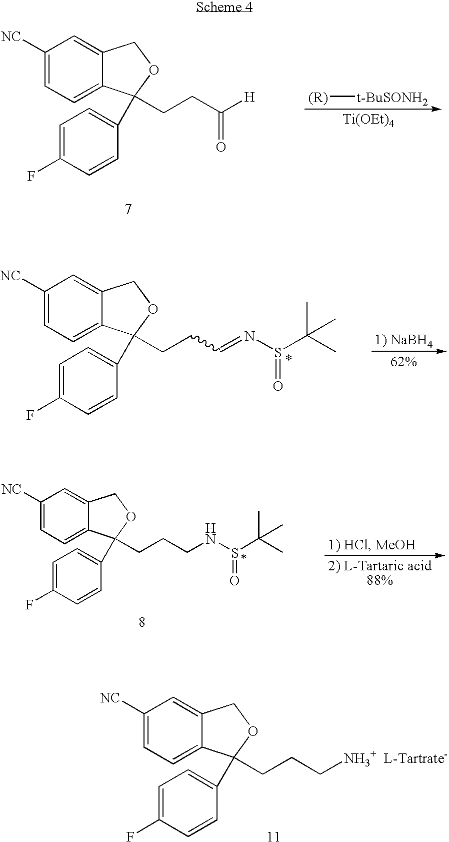

Another embodiment of the invention encompasses a method for preparing enantiomerically enriched didesmethylcitalopram by derivatizing racemic didesmethylcitalopram with BOC-anhydride, followed by column chromatography resolution of the racemic BOC-didesmethylcitalopram as shown in Scheme 5. Subsequent acidic hydrolysis of the BOC-derivatized enantiomers yields enantiomerically enriched didesmethylcitalopram. The preferred separation conditions are CHIRALCEL OD column with methanol as eluent.

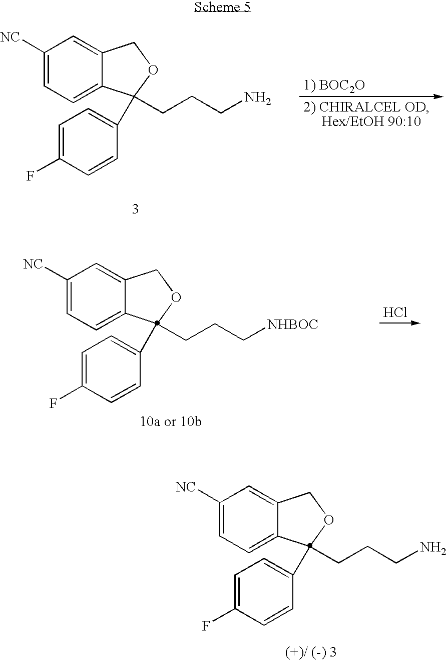

Another embodiment of the invention encompasses a versatile method for preparing enantiomerically enriched metabolites of citalopram by contacting aldehyde 7 with a variety of reagents as shown in Scheme 6.

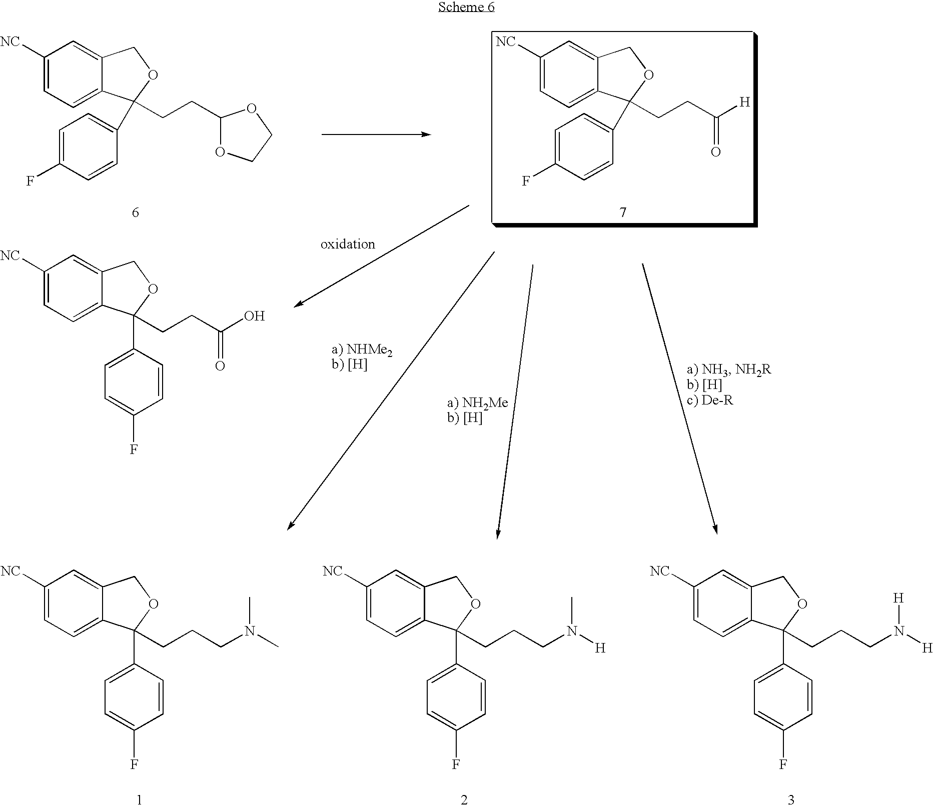

In an embodiment of the present invention, the compositions used in this invention selectively inhibit serotonin reuptake over reuptake of other monoamine neurotransmitters. In one embodiment, the compositions selectively inhibit serotonin reuptake over reuptake of dopamine or norepinephrine. In a still further preferred embodiment, the compositions used in this invention have IC₅₀'s for inhibition of serotonin reuptake that are more than two orders of magnitude lower than the corresponding IC₅₀ for inhibition of dopamine or norepinephrine reuptake.

One embodiment of the invention encompasses a method of treating a subject in need of such treatment with a therapeutically effective amount of enantiomerically pure (−)-desmethylcitalopram, or enantiomerically enriched (+) didesmethylcitalopram, or (−)-didesmethylcitalopram, a combination thereof, or a pharmaceutically acceptable salt, solvate, or clathrate thereof. The aforementioned citalopram metabolites can be used to treat serotonergic dysfunctions while exhibiting longer half lives than citalopram. While it is possible for a compound of the present invention to be administered alone, it is preferable to administer the compound as a pharmaceutical formulation (composition). The composition of the invention may be formulated for administration in any convenient way for use in human or veterinary medicine. In certain embodiments, the compound included in the pharmaceutical preparation may be active itself, or may be a prodrug of (−)-desmethylcitalopram, (+)-didesmethylcitalopram, or (−)-didesmethylcitalopram, which can be converted to an active compound in a physiological setting.

Thus, another aspect of the present invention provides pharmaceutically acceptable compositions comprising a therapeutically effective amount of one or more of the compounds described herein, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intrarectally, for example, as a pessary, cream or foam. However, in certain embodiments the subject compounds may be simply dissolved or suspended in sterile water. In certain embodiments, the pharmaceutical preparation is non-pyrogenic, i.e., does not substantially elevate the body temperature of a patient.

One embodiment of the invention is a pharmaceutical dosage form comprising a therapeutically effective amount of enantiomerically pure desmethyl or enantiomerically enriched didesmethyl metabolite of citalopram or a pharmaceutically acceptable salt, solvate, or clathrate or pharmaceutical excipient thereof. In one preferred embodiment, the dosage form is a tablet or a capsule or oral solution. In one embodiment, the dosage is adapted for intravenous infusion, transdermal delivery or oral delivery. In one embodiment a therapeutically effective amount of the dosage is from about 10 mg to about 500 mg. In one embodiment, the dosage is from about 25 mg to about 250 mg. In a still further preferred embodiment, the dosage is from about 50 mg to about 150 mg.

One embodiment of the invention is a method for treating affective disorders and reducing the clinical symptoms associated with affective disorders. In certain embodiments the particular affective disorders are comprised of reactive depression, endogenous depression and manic depression, anxiety disorders, attention deficit disorder, attention deficit disorder with hyperactivity, bipolar and manic conditions, bulimia, obesity or weight gain, narcolepsy, chronic fatigue syndrome, seasonal affective disorder, premenstrual syndrome, substance addiction or abuse, and nicotine addiction. In other embodiments the clinical symptoms include dysphoric mood or pervasive loss of interest or pleasure, accompanied by a number of the following symptoms: sleep and appetite disturbances, loss of energy, diminishment of sex drive, onset of body aches or pains, memory loss, inability to make decisions, feelings of self-reproach or excessive or inappropriate guilt, suicidal thoughts, and reduced ability to concentrate.

Thus, the invention encompasses a method of treating CNS affective disorders in a subject which comprises administering to a subject in need of such treatment a therapeutically effective amount of a citalopram metabolite, or a mixture or a pharmaceutically acceptable salt, solvate, or clathrate thereof.

Another embodiment of the invention encompasses method for treating cerebral function disorders. Such disorders include, but are not limited to, amnesia, dementia, Alzheimer's type dementia, psychosis, sleep disorders, post-traumatic stress disorders, borderline personality disorder, trichotillomania, or panic disorder. Cerebral function disorders may be induced by factors including, but not limited to, cerebrovascular diseases such as cerebral infarction, cerebral venous thrombosis, head injuries, and the like, and where symptoms include disturbances of the consciousness, senile dementia, coma, lowering of attention, speech disorders, and the like. Thus, the invention encompasses a method of treating a cerebral function disorder in a subject which comprises administering to a subject in need of such treatment a therapeutically effective amount of a citalopram metabolite, or a mixture or a pharmaceutically acceptable salt, solvate, or clathrate thereof.

Another embodiment of the invention is a method for treating vascular disorders which comprises administering to a subject in need of such treatment a therapeutically effective amount of a citalopram metabolite, or a mixture or a pharmaceutically acceptable salt, solvate, or clathrate thereof.

Another embodiment of the invention is a method for treating sexual dysfunction such as, but not limited to, premature ejaculation or erectile dysfunction, which comprises administering to a subject in need of such treatment a therapeutically effective amount of a citalopram metabolite, or a mixture or a pharmaceutically acceptable salt, solvate, or clathrate thereof.

Another embodiment of the invention is a method for treating premenstrual syndrome, which comprises administering to a subject in need of such treatment a therapeutically effective amount of a citalopram metabolite, or a mixture or a pharmaceutically acceptable salt, solvate, or clathrate thereof.

Another embodiment of the invention is a method for treating anxiety, which comprises administering to a subject in need of such treatment a therapeutically effective amount of a citalopram metabolite, or a mixture or a pharmaceutically acceptable salt, solvate, or clathrate thereof.

Another embodiment of the invention is a method for treating eating disorders, including but not limited to bulimia and anorexia, which comprises administering to a subject in need of such treatment a therapeutically effective amount of a citalopram metabolite, or a mixture or a pharmaceutically acceptable salt, solvate, or clathrate thereof.

Another embodiment of the invention is a method for treating obsessive compulsive disease, which comprises administering to a subject in need of such treatment a therapeutically effective amount of a citalopram metabolite, or a mixture or a pharmaceutically acceptable salt, solvate, or clathrate thereof.

Another embodiment of the invention is a method of treating, preventing or alleviating one or more symptoms caused by partial withdrawal form tobacco or nicotine, which comprises administering to a subject in need of such treatment a therapeutically effective amount of a citalopram metabolite, or a mixture or a pharmaceutically acceptable salt, solvate, or clathrate thereof.

Another embodiment of the invention is a method of treating, preventing cerebrovascular disorder which comprises administering to a subject in need of such treatment a therapeutically effective amount of a citalopram metabolite, or a mixture or a pharmaceutically acceptable salt, solvate, or clathrate thereof. In certain embodiments the disorder results from cerebral infarction, cerebral hemorrhage, cerebral arteriosclerosis, subarachnoid hemorrhage, cerebral thrombosis, or cerebral embolism. In other embodiments the disorder is ischemia, amnesia associated with ischemia, or vascular or multi infarct dementia.

Another embodiment of the invention is a method of treating, preventing vascular disorder which comprises administering to a subject in need of such treatment a therapeutically effective amount of a citalopram metabolite, or a mixture or a pharmaceutically acceptable salt, solvate, or clathrate thereof. In certain embodiments the disorder is selected form myocardial infarction, angina, stroke, pulmonary embolism, transient ischemic attack, deep vein thrombosis, thrombotic re-occulusion subsequent to a coronary intervention procedure, heart surgery or vascular surgery, peripheral vascular thrombosis, Syndrome X, heart failure, or a disorder in which a narrowing of at least one coronary artery occurs. In certain embodiments, the present invention provides a method for treating a subject at risk for vascular event, disease, or disorder so as to reduce the risk of occurrence of the vascular event, wherein the vascular event is comprised of the aforementioned manifestations of a vascular disorder.

Another embodiment of the invention is a method of co-administration of the pharmaceutical compositions described herein with another agent selected from an antipsychotic, anticonvulsant, mood stabilizing agent, or a central nicotine stimulating agent. Examples of drugs amenable for co-treatment include clozapine, therophylline, warfarine, imipramine, mephenyloin, sparteine, amitriptyline, carbamazepine, triazolam, benzodiazepine, risperidone, gabapentin, and lamotrigine. Alternatively, the co-administered agent may be a substrate for a cytochrome P450 enzyme selected from CYP1A2, CYP2C9, CYP2C19, CYP2D6, or CYP3A4.

Enantiomerically pure or enriched (−)-desmethylcitalopram, or enantiomerically enriched didesmethylcitalopram is intended to provide an improvement in the treatment of such disorders because they exhibit longer half lives than citalopram.

Another aspect of the present invention is a method for conducting a pharmaceutical business, comprising:

a. manufacturing a pharmaceutical composition disclosed herein; and

b. marketing to healthcare providers the benefits of using the composition in the treatment one or more disorders, dysfunctions, or diseases for which serotonin reuptake inhibition is therapeutically beneficial.

In one embodiment, the present invention provides a method for conducting a pharmaceutical business, comprising:

a. providing a distribution network for selling a pharmaceutical composition disclosed herein; and

b. marketing to healthcare providers the benefits of using the composition in the treatment one or more disorders, dysfunctions, or diseases for which serotonin reuptake inhibition is therapeutically beneficial.

In another embodiment, the present invention provides a method for conducting a pharmaceutical business, comprising:

a. determining an appropriate formulation and dosage of a pharmaceutical composition disclosed herein, singly or in combination with one or more therapeutic agent(s) selected from antipsychotics, anticonvulsants, psychostimulants, mood stabilizing agents, or central nicotine stimulating agents;

b. conducting therapeutic profiling of formulations identified in step (a), for efficacy and toxicity in animals; and

c. providing a distribution network for selling a preparation or preparations identified in step (b) as having an acceptable therapeutic profile.

In certain embodiments, the pharmaceutical business method disclosed in the present invention includes an additional step of providing a sales group for marketing the preparation to healthcare providers. Another embodiment of the present invention provides a method for conducting a pharmaceutical business, comprising:

a. determining an appropriate formulation and dosage of a pharmaceutical composition disclosed herein, singly or in combination with one or more therapeutic agent(s) selected from antipsychotics, anticonvulsants, psychostimulants, mood stabilizing agents, or central nicotine stimulating agents; and

b. licensing, to a third party, the rights for further development and sale of the formulation.

D. Pharmacological Formulations

In another aspect, the present invention provides pharmaceutical compositions. The composition for use in the subject method may be conveniently formulated for administration with a biologically acceptable medium, such as water, buffered saline, polyol (for example, glycerol, propylene glycol, liquid polyethylene glycol and the like) or suitable mixtures thereof. The optimum concentration of the active ingredient in the chosen medium can be determined empirically, according to procedures well known to medicinal chemists. As used herein, “biologically acceptable medium” includes any and all solvents, dispersion media, and the like which may be appropriate for the desired route of administration of the pharmaceutical preparation. The use of such media for pharmaceutically active substances is known in the art. Except insofar as any conventional media or agent is incompatible with the activity of the serotonin reuptake inhibition, its use in the pharmaceutical preparation of the invention is contemplated. Suitable vehicles and their formulation inclusive of other proteins are described, for example, in the book Remington's Pharmaceutical Sciences (Remington's Pharmaceutical Sciences. Mack Publishing Company, Easton, Pa., USA 1985). These vehicles include injectable “deposit formulations”.

Pharmaceutical formulations of the present invention can also include veterinary compositions, e.g., pharmaceutical preparations of the serotonin reuptake inhibitor suitable for veterinary uses, e.g., for the treatment of livestock or domestic animals, e.g., dogs.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a drug at a particular target site.

The preparations of the present invention may be given orally, parenterally, topically, or rectally. They are of course given by forms suitable for each administration route. For example, they are administered in tablets or capsule form, by injection, inhalation, eye lotion, ointment, suppository, controlled release patch, etc.; administration by injection, infusion or inhalation; topical by lotion or ointment; and rectal by suppositories. Oral and topical administrations are preferred.

The phrases “parenteral administration” or “administered parenterally” as used herein mean modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally, and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms such as described below or by other conventional methods known to those of skill in the art.

Actual dosage levels of the active ingredients in the pharmaceutical compositions of this invention may be varied so as to obtain an amount of the active ingredient effective to achieve the desired therapeutic response for a particular patient, composition, and mode of administration without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular composition employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous, intracerebroventricular and subcutaneous doses of the compounds of this invention for a patient will range from about 0.0001 to about 100 mg per kilogram of body weight per day.

If desired, the effective daily dose of the active compound may be administered daily in two, three, four, five, six or more sub-doses administered separately at appropriate intervals, optionally, in unit dosage forms. A physician or veterinarian having ordinary skill in the art can readily determine the total duration of the treatment regime.

The term “treatment” is intended to encompass also prophylaxis, therapy and cure.

The patient receiving this treatment is any animal in need, including primates, in particular humans, and other mammals such as equines, cattle, swine and sheep; and poultry and pets in general.

The compound of the invention can be administered as such or in admixtures with pharmaceutically acceptable and/or sterile carriers and can also be administered in conjunction with other antimicrobial agents such as penicillins, cephalosporins, aminoglycosides and glycopeptides. Conjunctive therapy, thus includes sequential, simultaneous and separate administration of the active compound in a way that the therapeutical effects of the first administered drug are not entirely past when the subsequent drug is administered.

The phrase “therapeutically effective amount” as used herein means that amount of a compound, material, or composition comprising a compound of the present invention which is effective for producing some desired therapeutic effect by inhibition of serotonin reuptake in at least a sub-population of cells in an animal and thereby blocking the biological consequences of that pathway in the treated cells, at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject antagonists from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its metabolites, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (H) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As set out above, certain embodiments of the present composition may contain a basic functional group, such as amino or alkylamino, and are, thus, capable of forming pharmaceutically acceptable salts with pharmaceutically acceptable acids. The term “pharmaceutically acceptable salts” in this respect refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, besylate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19)

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Pharmacological dosages or formulations of the present invention include those suitable for oral, nasal, topical (including buccal and sublingual), rectal, vaginal and/or parenteral administration. The dosages may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

It is known that sterols, such as cholesterol, will form complexes with cyclodextrins. Thus, in preferred embodiments, where the inhibitor is a steroidal alkaloid, it may be formulated with cyclodextrins, such as α-, β- and γ-cyclodextrin, dimethyl-βcyclodextrin and 2-hydroxypropyl-β-cyclodextrin.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active ingredient.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose metabolites, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the composition in the proper medium. Absorption enhancers can also be used to increase the flux of the composition across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The addition of the active compound of the invention to animal feed is preferably accomplished by preparing an appropriate feed premix containing the active compound in an effective amount and incorporating the premix into the complete ration.

Alternatively, an intermediate concentrate or feed supplement containing the active ingredient can be blended into the feed. The way in which such feed premixes and complete rations can be prepared and administered are described in reference books (such as “Applied Animal Nutrition”, W.H. Freedman and CO., San Francisco, U.S.A., 1969 or “Livestock Feeds and Feeding” O and B books, Corvallis, Ore., U.S.A., 1977).

### EXAMPLES

### Example 1

Synthesis of 4-[3-[1,3]-dioxolan-2-yL-1-(4-fluorophenyl)-1-hydroxypropyl]-3-hydroxymethylbenzonitrile (5): To a suspension of 1-oxo-1,3-dihydro-isobenzofuran-5-carbonitrile 5 (15 g) in THF (50 mL, anhydrous) at 5-10° C. under argon was added 4-flurophenylmagnisum bromide in ethyl ether (50 ml, 2 M). The reaction mixture was warmed to room temperature and stirred for 5 h. At that time, a second Grignard reagent [prepared from 2-(2-bromoethyl)-[1,3]-dioxolane (25 g) with Mg powder in THF] was added at room temperature. The reaction mixture was stirred at room temperature for 14 h. The reaction mixture was then quenched at 0° C. with aqueous ammonium chloride. The organic phase was separated and washed with water (50 mL), and concentrated to give a crude product. It was purified by flash chromatography (EtOAc: Hexane 1:1) to give 17 g of the titled product 5. ¹H NMR (CDCl₃, δ): 8 1.54-1.66 (m, 1H), 1.75-1.88 (m, 1H), 2.18-2.30 (m, 1H), 2.36-2.47 (m, 1H), 356 (broad s, 1H), 3.80-4.00 (m, 4H), 4.10-4.17 (d, J=8 Hz, 1H), 4.22-4.30 (d, J=8 Hz, 1H), 4.86 (t, J=3 Hz, 1H), 5.50 (broad s, 1H), 6.8-7.10 (m, 2H), 7.16-7.26 (m, 2H), 7.50-7.70 (m, 3H). ³C NMR(CDCl₃, δ): 8 27.2, 35.8, 63.1, 64.9, 77.8, 103.4, 111.4, 114.7, 115.0, 118.3, 127.1, 127.6, 127.3, 131.1, 134.7, 141.1, 149.7, 159.9, 163.2.

### Example 2

Synthesis of 1-(2-[1,3]-dioxolan-2-yL-ethyl)-1-(4-fluorophenyl)-1,3-dihydro-isobenzofuran-5-carbonitrile (6): 4-[3-[1,3]-dioxolan-2-yL-1-(4-fluorophenyl)-1-hydroxypropyl]-3-hydroxymethylbenzonitrile (15 g) was dissolved in dichloromethane (150 mL) at room temperature, followed by addition of triethylamine (25 mL). The reaction mixture was cooled to 5-10° C. and stirred for 5 min, followed by addition of methane sulfonylchloride (8.4 g). The reaction mixture was warmed to room temperature and stirred for 10 min. It was quenched with 2% NaOH (100 mL) while maintained to 10-20° C. The organic phase was separated and was water, concentrated to give 19.2 g crude product. ¹H NMR (CDCl₃, δ): 1.42-1.57 (m, 1H), 1.63-1.76 (m, 1H), 2.20-2.38 (m, 2H), 3.78-3.96 (m, 4H), 4.84 (t, J=3 Hz, 1H), 5.18 (m, 2H), 7.00 (m, 2H), 7.38-7.50 (m, 4H), 7.60 (m, 1H). 2 was separated to its enantiomer by CHIRALCEL OD=column using Methanol as eluent. Enantiomer 8a, 6.67 min; Enantiomer 8b, 8.30 min.

### Example 3

Synthesis of 1-(4-fluorophenyl)-1-(3-Oxopropyl)-1,3-dihydro-isobenzofuran-5-carbonitrile (7): 1-(2-[1,3]-dioxolan-2-yL-ethyl)-1-(4-fluorophenyl)-1,3-dihydro-isobenzofuran-5-carbonitrile (6 g) was dissolved in a mixture of acetone (100 mL) and aqueous HCl (5 N, 30 mL). The reaction mixture was stirred at room temperature for 60 h, and concentrated to remove acetone. The aqueous solution was extracted with ethyl acetate (40 mL), and concentrated to give a crude oil. It was dissolved in acetone (80 mL) and aqueous HCl (SN, 25 mL), stirred for 3 h, and concentrated to remove acetone. The aqueous solution was extracted with ethyl acetate (40 mL), washed with water, and concentrated to give the pure product 7 (5.6 g). ¹H NMR (CDCl₃, δ): 2.22-2.64 (m, 4H), 5.14 (s, 2H), 6.94-7.06 (m, 2H), 7.40-7.63 (m, 5H), 9.70 (s, 1H).

### Example 4

Synthesis of 2-MethyL-propane-2-sulfinic acid [3-[5-cyano-L-(4-flurophenyl)-1,3-dihydro-isobenzofuran-1-yl]-propyl]-amide (8): 1-(4-fluorophenyl)-1-(3-Oxopropyl)-1,3-dihydro-isobenzofuran-5-carbonitrile (3.0 g) was dissolved in THF (20 mL), followed by addition of (−)-tert-butylsulfinamide (1.5 g), and Ti(OEt)₄ (20 mL, Aldrich) in EtOH. The reaction mixture was stirred at room temperature for 10 min, and 55° C. for 1 h. The reaction mixture was cooled to 5-10° C., was added brine (50 μL), and EtOAc (150 μL). The reaction mixture was stirred at room temperature for 10 min and filtered. The EtOAc layer in the filtrate was separated and washed with brine and concentrated to give a crude oil. It was dissolved in THF (20 ml), cooled to 5-10° C. and added NaBH₄ (1.6 g) Methanol (10 mL). The reaction mixture was stirred for 14 h, quenched with water at 5-10° C., extracted with EtOAc (100 mL). The extract was washed with brine and concentrated to give the crude product (3.5 g). It was passed through a silica gel column using EtOAc and hexane (8:2) to give the pure product (2.5 g) as a mixture of possible diastereoisomers. ¹H NMR (CDCl₃, δ): 1.19 (s, 9H), 1.40-1.60 (m, 2H), 2.10-2.30 (m, 2H), 3.05-3.30 (m, 3H), 5.17 (m, 2H). 7.00 (m, 2H), 7.40-7.60 (m, 5H). ¹³C NMR (CDCl₃, δ): 22.4, 25.6, 38.1, 45.4, 55.4, 71.1, 90.7, 111.6, 115.1, 115.4, 118.4, 122.6, 125.1, 126.5, 126.6, 131.8, 139.1, 140.1, 149.0, 160.2, 163.5.

### Example 5

Synthesis of 3-[5-cyano-L-(4-flurophenyl)-1,3-dihydro-isobenzofuran-1-yl]-propyl amine (Didesmethylcitalopram) (3): 2-MethyL-propane-2-sulfinic acid [3-[5-cyano-L-(4-flurophenyl)-1,3-dihydro-isobenzofuran-1-yl]-propyl]-amide (2.0 g) was dissolved in Methanol (35 mL) at room temperature, followed by addition of HCl (10%, 20 mL). The reaction mixture was stirred at room temperature for 16 h. The reaction mixture was concentrated to remove Methanol and added TBME (100 mL), water (50 mL), and aqueous potassium carbonate till basic. The organic phase was separated and washed with water (20 mL), brine (20 mL), and dried over sodium sulfate, concentrated to give an oil (1.3 g) as free base. ¹H NMR (CDCl₃, δ): 1.23 (broad s, 2H), 1.21.30-1.43 (m, 2H), 2.08-2.25 (m, 2H), 2.63-70 (t, J=7 Hz, 2H), 5.10-5.20 (m, 2H). 7.00 (m, 2H), 7.38-7.60 (m, 5H).

### Example 6

Synthesis of BOC-Didesmethylcitalopram (10): To a solution of 3 (3.3 g) in dichloromethane (40 mL) was added triethylamine (10 mL) at room temperature, followed by addition of BOC anhydride (5.0 g). The reaction mixture was stirred for 1 h. The reaction mixture was then concentrated to a residue, which was passed through a flash silica gel column (EtOAc:Hexane 3:7) to give 3.5 product. ¹H NMR (CDCl₃, δ): 1.30-1.50 (m, 2H), 1.41 (s, 9H), 2.06-2.28 (m, 2H), 3.15 (m, 2H), 4.54 (s, 11-1), 5.10-5.21 (m, 2H), 7.00-7.06 (m, 2H), 7.30-7.42 (m, 3H), 7.50 (s, 1H), 7.60 (d, J=8 Hz, 1H). The racemic product 10 was separated by CHIRALCEL OD column (Hexane:Ethanol 90:10) to give 10a (>99% ee, 7.20 min) and 10b (>99% ee, 8.4 min).

### Example 7

Synthesis of (+)-Didesmethylcitalopram [(+)-3]: Compound 10a (1.3 g) was dissolved in TFA (10 mL). It was stirred at room temperature for 1 h, and was concentrated to give a residue, which was added water (15 mL) and EtOAc (30 mL) and aqueous potassium carbonate till basic. Organic phase was separated and washed with water, brine, and concentrated to give the product (0.96 g, >95%). [(+)-3]. [α]=+5.5° (C=1, Methanol). (−)-3 was prepared from 10b. Their H NMR spectra are the same as the racemate.

### Example 8

Synthesis of Didesmethylcitalopram L-tartrate (11): To a solution of the didesmethylcitalopram free base (1.3 g) in Methanol (20 mL) was added a solution of L-tartaric acid (0.6 g) in water (5 mL). The reaction mixture was stirred for 30 min, and concentrated to give a white solid (1.8 g). ¹H NMR (DMSO-d₆, δ): 1.26-1.60 (m, 2H), 2.24-2.30 (m, 2H), 2.70-2.80 (m, 2H), 4.03 (s, 2H), 5.10-5.25 (m, 2H), 7.15-7.20 (tm, 2H), 7.58-7.64 (m, 2H), 7.73-7.81 (m, 3H), 7.0-7.9 (broad, 4H). ¹³C NMR (DMSO-d₆, δ): 23.0, 17.8, 72.7, 91.1, 111.3, 115.8, 116.0, 119.5, 123.9, 126.4, 127.6, 127.7, 132.8, 140.6, 140.9, 149.7, 160.4, 163.7, 175.4. M+ 296.9.

### Example 9

Synthesis of 1-(4-fluorophenyll)-1-(3-methylamino-propyll)-1,3-dihydroisobenzofuran-5-carbonitrile) (Desmethylcitalopram) (2): 1-(4-fluorophenyl)-1-(3-Oxopropyl)-1,3-dihydro-isobenzofuran-5-carbonitrile (3.0 g) was dissolved in aqueous methylamine (10 mL, 40%). The reaction mixture was stirred for 2 h at room temperature. It was extracted with tert-butyl methyl ether (50 mL). The organic layer was washed with brine and concentrated to give a crude corresponding imine. It was dissolved in methanol (30 μL), and treated with sodium borohydride (2 g) at 10-20° C. for 30 min The reaction mixture was then quenched with aqueous ammonium chloride (50 mL) and 5 N HCl till acidic. The reaction mixture was stirred for 10 min, followed by addition of potassium carbonate till basic (pH>9.5). This reaction mixture was then extracted with ethyl acetate (2×100 mL). The extracts were combined and washed with water, brine and concentrated to give the crude product. It was purified by flash chromatography (EtOAc:MeOH:Et₃N=8:1:1) to give 1.4 g titled product as free base. ¹H NMR (CDCl₃, δ): 1.30-1.66 (m, 2H), 2.10-2.30 (m, 2H), 2.35 (s, 3H), 2.53-2.60 (m, 2H), 5.10-5.25 (m, 2H), 6.94-7.05 (m, 2H), 7.40-7:62 (m, 5H). ‘3C NMR 8. 23.9, 35.9, 38.8, 51.4, 71.2, 90.9, 111.5, 115.0, 115.3, 118.5, 122.7, 125.1, 126.6, 126.7, 131.7, 139.4, 140.2, 149.2, 160.2, 163.5. M+310.9. (+)-Desmethylcitalopram [(+)-2] was prepared similarly from enantiomerically pure 8b. [α]_{—}+6.0 (c=2, Methanol). (−)-Desmethylcitalopram [(−)-2] was prepared from enantiomer 8a. [α]=−5.3° (c=2, Methanol).

### Example 10

Synthesis of Desmethylcitalopram L-tartrate (12): To a desmethylcitalopram (1.2 g) solution in Methanol (15 mL) was added a solution of L-tartaric acid (0.58 g) in water (2 mL). The reaction mixture was stirred at room temperature for 1 h. The solvent was then removed to give the final salt. ¹H NMR (DMSO-d₆, δ): 1.26-1.60 (m, 2H), 2.24-2.30 (m, 2H), 2.35 (s, 3H), 2.70-2.80 (m, 2H), 4.03 (s, 2H), 5.10-5.25 (m, 2H), 7.15-7.20 (tm, 2H), 7.58-7.64 (m, 2H), 7.73-7.81 (m, 3H), 7.0-7.9 (broad, 4H). ¹³C NMR (DMSO-d₆, δ): 21.4, 33.0, 37.8, 48.8, 71.9, 72.8, 91.1, 111.3, 115.8, 116.0, 119.5, 123.9, 126.4, 127.7, 132.8, 140.6, 140.8, 149.6, 159.8, 163.0, 175.5; M+ 310.9

### Example 11

Pharmacology: Racemic citalopram, and single enantiomers of desmethylcitalopram and didesmethylcitalopram were compared for their ability to inhibit the reuptake of radiolabeled serotonin into synaptosomes prepared from various regions of rat brains. The ability of the same compounds to inhibit the reuptake of norepinephrine (NE) and dopamine into similarly prepared synaptosomes was also measured to assess the selectivity of racemic citalopram, and the (+) and (−) enantiomers of its metabolites, for inhibition of serotonin reuptake vis-a-vis the reuptake of NE and dopamine. The five compounds were also evaluated for their ability to inhibit specific binding of radiolabeled ligands to the nonselective muscarinic receptor.

IC₅₀ values (concentration inhibiting neurotransmitter reuptake or specific binding by 50%) were calculated by regression analysis of the inhibition curves (Table 1).

TABLE 1
Inhibition of Specific Muscarine Receptor Bindings and
Serotonin, Dopamine, and Norepinephrine
Reuptake into Synaptosomes by Racemic Citalopram,
Enantiomeric Desmethylcitalopram and
Didesmethylcitalopram.
IC₅₀ values (in nM) for racemic citalopram and enantiomeric
metabolites on functional monoamine uptake

| | 5-HT uptake | NE uptake | DA uptake |
|---|---|---|---|
| Test compounds | | | |
| (−)-Didesmethylcitalopram | 130 | 1,300 | 2,700 |
| (+)-Didesmethylcitalopram | 180 | 3,300 | 11,000 |
| (−) Desmethylcitalopram | 110 | 1,700 | 9,400 |
| (+) Desmethylcitalopram | 5.8 | 4,100 | 7,600 |
| Racemic Citalopram | 2.2 | 4,900 | 13,000 |
| Reference compounds | | | |
| protriptyline | | 0.0010/ | |
| | | 0.0019/ | |
| | | 0.0041 | |
| GBR 12909 | | | 0.0031 |
| imipramine | 0.025 | | |

Within the serotonin transporter system, values and changes from the parent citalopram to its demethylated metabolites correlated closely between species (Table 2)

TABLE 2
IC₅₀ and Kᵢ values (in nM) determined for the racemic citalopram and
enantiomeric metabolites at the human NE and DA transporters

| 5-HT Transporter | | | | NE | | DA | |
|---|---|---|---|---|---|---|---|
| Rat | | Human | | Transporter | | Transporter | |
| IC₅₀ | Kᵢ | IC₅₀ | Kᵢ | IC₅₀ | Kᵢ | IC₅₀ | Kᵢ |

Test
Compounds

| | | | | | | | | |
|---|---|---|---|---|---|---|---|---|
| (−)-Didesmethylcitalopram | 27 | 5.5 | 14 | 5.8 | 5,520 | 5,120 | 38,900 | 17,800 |
| (+)-Didesmethylcitalopram | 19 | 3.9 | 219 | 90 | 24,200 | 22,400 | 54,700 | 25,000 |
| (−)-Desmethyl | 11 | 2.3 | 42 | 17 | 815 | 756 | 37,600 | 17,100 |
| citalopram | | | | | | | | |
| (+)-Desmethyl | 1.3 | 0.27 | 20 | 8.2 | 10,300 | 9,570 | 27,700 | 12,700 |
| citalopram | | | | | | | | |
| Racemic | 1.4 | 0.30 | 9.4 | 3.9 | 4,600 | 4,270 | 38,800 | 17,700 |
| citalopram | | | | | | | | |
| Reference Compounds | | | | | | | | |
| Imipramine | | | 11 | 4.6 | | | | |
| Zimelidine | 57 | 12 | | | | | | |
| protriptyline | | | | | 5.2/6.3 | 4.8 | | |
| GBR12909 | | | | | | | 8.1/ | 3.7 |
| | | | | | | | 6.3/2.5 | |

As reported previously, citalopram showed high selectivity for the serotonin transporter in its inhibition of monoamine neurotransmitter reuptake. The same trend continued for (+) enantiomer of desmethylcitalopram. Contrary to prior teachings, however, the IC₅₀ for the (−) enantiomers or desmethyl- and didesmethylcitalopram as well as the (+) enantiomer of didesmethylcitalopram were within the micromolar range. On the basis of these results, it appears that both desmethyl and didesmethyl forms of citalopram retain significant serotonin reuptake inhibitory activity.

1. A compound having a structure depicted in Formula I, 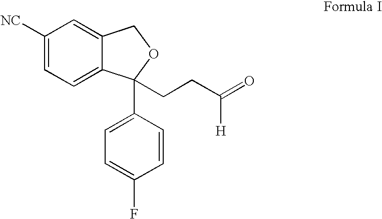
wherein the compound is a racemic, enantiomerically enriched, or optically pure form of the compound.

2. A compound having a structure depicted in Formula II, 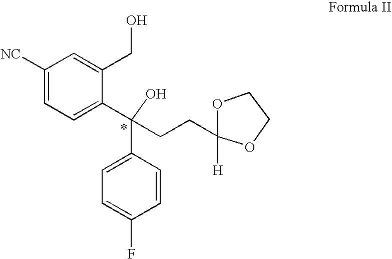
wherein the compound is a racemic, enantiomerically enriched, or optically pure form of the compound.

3. A method for synthesizing (−)-desmethylcitalopram, comprising:
a. reacting 5-cyanophthalide with 4-fluorophenyl magnesium bromide in the presence of a chiral ligand, followed by reaction with a second Grignard reagent prepared by reacting 2-bromoethyldioxolane with magnesium, to afford the (−)-tertiary alcohol shown in Formula III; 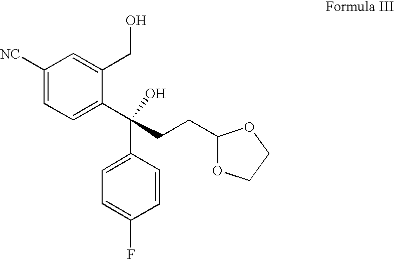
b. reacting the (−)-tertiary alcohol shown in Formula III with mesyl chloride followed by acidic treatment to afford the (−)-aldehyde shown in Formula IV; and 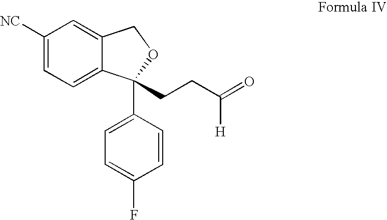
c. reducing the structure in Formula IV with sodium borohydride in the presence of methylamine.

4. The method of claim 3, wherein the resultant amine is isolated as is or is subsequently reacted with an acid to form a salt.

5. The method of claim 4, wherein the acid used is D-tartaric acid, L-tartaric acid, HCl or HBr.

6. The method of claim 3, wherein column chromatography with chiral solid support is used to separate the enantiomers of final or intermediate products.

7. The method of claim 3, wherein the enantiomeric enrichment achieved is greater than 80%.

8. The method of claim 3, wherein the enantiomeric enrichment achieved is greater than 90%.

9. The method of claim 3, wherein the enantiomeric enrichment achieved is greater than 95%.

10. The method of claim 3, wherein the enantiomeric enrichment achieved is greater than 99%.

11. A method of synthesizing (−)-didesmethylcitalopram or (+)-didesmethylcitalopram, said method comprising: 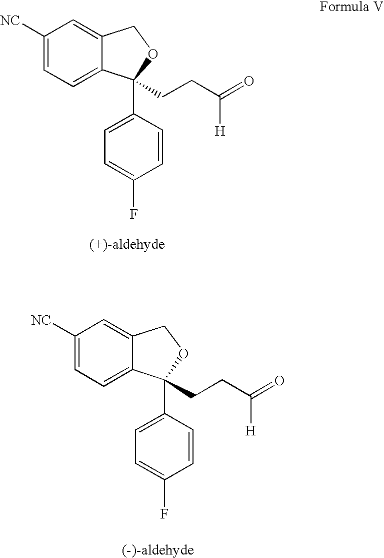
a. reacting either the (−)-aldehyde or (+)-aldehyde shown in Formula V with either (+)-tert-butylsulfinamide or (−)-tert-butylsulfinamide in the presence of Ti(OEt)₄ to give sulfinimine shown in Formula VI 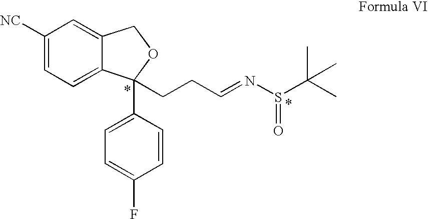
wherein the compound is optically pure or enantiomerically enriched at each stereocenter designated with asterisks in Formula VI; and
b. reducing the sulfinimine in Formula VI to an amine with sodium borohydride.

12. The method of claim 11, wherein the resultant amine is isolated as is or is subsequently reacted with an acid to form a salt.

13. The method of claim 12, wherein the acid used is D-tartaric acid, L-tartaric acid, HCl or HBr.

14. The method of claim 11, wherein column chromatography with chiral solid support is used to separate the enantiomers of final or intermediate products.

15. The method of claim 11, wherein the enantiomeric enrichment achieved is greater than 80%.

16. The method of claim 11, wherein the enantiomeric enrichment achieved is greater than 90%.

17. The method of claim 11, wherein the enantiomeric enrichment achieved is greater than 95%.

18. The method of claim 11, wherein the enantiomeric enrichment achieved is greater than 99%.

19. A pharmaceutical composition comprising a pharmaceutically acceptable excipient and enantiomerically pure (−)-desmethylcitalopram, or enantiomerically enriched (−)-didesmethylcitalopram or (+)-didesmethylcitalopram, or any combination thereof, or a pharmaceutically acceptable salt, solvate or clathrate thereof.

20. A method of treating one or more disorders, dysfunctions or diseases for which serotonin reuptake inhibition is therapeutically beneficial, comprising administering to a subject a therapeutically effective amount of a composition of claim 19.

21. The method of claim 20, wherein the disorder comprises at least one of depression, an anxiety disorder, attention deficit disorder, attention deficit disorder with hyperactivity, bipolar and manic conditions, bulimia, obesity or weight gain, narcolepsy, chronic fatigue syndrome, seasonal affective disorder, premenstrual syndrome, substance addiction or abuse, and nicotine addiction.

22. The method of claim 20, wherein the treatment is for reducing clinical symptoms of affective disorders selected from dysphoric mood or pervasive loss of interest or pleasure, accompanied by a number of the following symptoms: sleep and appetite disturbances, loss of energy, diminishment of sex drive, onset of body aches or pains, memory loss, inability to make decisions, feelings of self-reproach or excessive or inappropriate guilt, suicidal thoughts, and reduced ability to concentrate.

23. The method of claim 20, wherein the disorder comprises reactive depression, endogenous depression, or manic depression.

24. The method of claim 20, wherein the disorder, dysfunction, or disease is selected from one or more of sexual dysfunction, eating disorders, substance abuse, cerebrovascular disorder, vascular disorder, obsessive-compulsive disease, anxiety, dementia, or canine affective aggression.

25. The method of claim 20, wherein the disorder, dysfunction, or disease comprises premature ejaculation or erectile dysfunction.

26. The method of claim 20, wherein the disorder, dysfunction, or disease comprises bulimia or anorexia nervosa.

27. The method of claim 20, wherein the treatment prevents or alleviates one or more symptoms caused by withdrawal or partial withdrawal from use of tobacco or nicotine.

28. A method of claim 20, wherein the disorder, dysfunction, or disease is a cerebrovascular disorder caused by cerebral infarction, cerebral hemorrhage, cerebral arteriosclerosis, subarachnoid hemorrhage, cerebral thrombosis, cerebral embolism, ischemic, amnesia, or multi infarct dementia.

29. The method of claim 20, wherein the disorder, dysfunction, or disease is a vascular disorder selected from myocardial infarction, angina, stroke, pulmonary embolism, transient ischemic attack, deep vein thrombosis, thrombotic re-occulusion subsequent to a coronary intervention procedure, heart surgery or vascular surgery, peripheral vascular thrombosis, Syndrome X, heart failure, or a disorder in which a narrowing of at least one coronary artery occurs.

30. A method of claim 29, wherein the subject is at risk for a vascular event, disease or disorder, and the treatment reduces the risk of occurrence of the vascular event.

31. A pharmaceutical kit comprising (i) a pharmaceutical composition of claim 19 and (ii) one or more therapeutic agent(s) selected from antipsychotics, anticonvulsants, psychostimulants, mood stabilizing agents, or central nicotine stimulating agents for co-administration with (i).

32. The pharmaceutical kit of claim 31, wherein the agent is a substrate for a cytochrome P450 enzyme selected from CYP1A2, CYP2C9, CYP2C19, CYP2D6, or CYP3A4.

33. The pharmaceutical kit of claim 31, wherein the agent is selected from clozapine, theophylline, warfarin, imipramine, mephenyloin, sparteine, amitriptyline, carbamazepine, triazolam, benzodiazepine, risperidone, gabapentin, or lamotrigine.

34. A pharmaceutical dosage form comprising a therapeutically effective amount of the pharmaceutical composition of claim 19.

35. The dosage form of claim 34, wherein said dosage form is a tablet or a capsule or oral solution.

36. The dosage form of claim 34, wherein said dosage form is adapted for intravenous infusion, transdermal delivery or oral delivery.

37. The dosage form of claim 34, wherein the therapeutically effective amount is ranges from 10 mg to 500 mg.

38. The dosage form of claim 34, wherein the therapeutically effective amount ranges from 25 mg to 250 mg.

39. The dosage form of claim 34, wherein the therapeutically effective amount ranges from 50 mg to 150 mg.
